(19) United States
(12) Patent Application Publication
(10) Pub. No.: US 2024/0230615 A1
(43) Pub. Date: Jul. 11, 2024

(54) METHOD FOR CHARACTERISING THE PRESENCE OF POLYMERS AND/OR QUANTIFYING POLYMERS IN A POROUS MEDIUM

(71) Applicant: IFP Energies nouvelles, RUEIL-MALMAISON (FR)

(72) Inventors: Maria-Fernanda ROMERO-SARMIENTO, RUEIL-MALMAISON CEDEX (FR); Herman RAVELOJAONA, RUEIL-MALMAISON CEDEX (FR); Daniel PILLOT, RUEIL-MALMAISON CEDEX (FR); Sebastien ROHAIS, RUEIL-MALMAISON CEDEX (FR)

(21) Appl. No.: 18/561,226

(22) PCT Filed: May 9, 2022
(86) PCT No.: PCT/EP2022/062455

§ 371 Date: Nov. 15, 2023

(30) Foreign Application Priority Data
May 17, 2021 (FR) FR2105123

(51) Int. Cl. G01N 33/24

(52) U.S. Cl. CPC G01N 33/241

(57) ABSTRACT

The invention concerns a method of characterizing a polymer (Pol) in a porous medium (Mil), wherein at least the following steps are carried out:
a) heating a porous medium sample (Mil), in an inert atmosphere, and continuously measuring a representative quantity of hydrocarbon compounds, a representative quantity of carbon monoxide and/or a representative quantity of carbon dioxide released,
b) heating a residue of said sample in an oxidizing atmosphere, and measuring a representative quantity of carbon monoxide and a representative quantity of carbon dioxide released,
c) comparing at least the quantities measured in steps a) and b) with reference parameters of a first polymer database (Pol),
d) determining the presence or the absence of a polymer (Pol) in porous medium (Mil) from the measurement comparison made in step c) and/or quantifying polymer (Pol) in porous medium (Mil).

### FIELD OF THE INVENTION

The invention concerns the field of characterization and quantification of polymers in a porous medium, notably in order to identify the pollution level of a sediment, of a soil of a natural environment such as a beach.

The invention also allows quantification of polymers in the porous medium in order to assess the level of environmental pollution.

The use of plastics (polymer-based materials) has been developed for many years in the world industry. Today, traces of these polymers can be found all over the world, notably in soils, rivers, seas and oceans. They are an increasing source of pollution.

Besides, these polymers can be found in the environment in microscopic form, invisible to the human eye, but it can still have a significant impact on the flora and fauna.

Furthermore, these polymers can transform in the environment, due to physical or chemical phenomena, and thus create compounds known as “neoformed” compounds, i.e. existing only because of the presence of polymers in the environment.

Today, characterization of the environment and notably of sands, soils and other underground or superficial geological formations is important in order to assess the current pollution level due to polymers and, on the other hand, to evaluate the evolution over time of this pollution in the future.

### BACKGROUND OF THE INVENTION

There are known plastics and polymer characterization methods that can be used to identify the presence of plastics and polymers in a natural environment such as sand or rocks: for example, differential scanning calorimetry, rheology testing, thermogravimetric analysis, dynamic mechanical analysis, pyrolysis-gas chromatography-mass spectrometry or Raman spectroscopy.

However, these methods are more or less complex and long.

Prior art also knows the ROCK-EVAL® device (IFP Energies nouvelles, France) developed by the applicant, notably described in documents FR-2,227,797 (U.S. Pat. No. 3,953,171) and FR-2,472,754 (U.S. Pat. No. 4,352,673). The ROCK-EVAL® device allows pyrolysis in an inert atmosphere (non-oxidizing, i.e. without the presence of oxygen) and oxidation, according to a sequence of predefined temperatures of a sample, for example a sedimentary rock sample. The pyrolysis oven cooperates with a device for detecting and measuring the quantity of hydrocarbon compounds of the pyrolyzed sample. The specific detection device comprises, for example, a flame ionization type detector conventionally used in gas chromatography analysis. The detector delivers a signal representative of the measured quantities of hydrocarbon products. This signal can be transmitted to computing, storage and display means wherein a specific software computes, displays and stores the various parameters representative of the characteristics of the hydrocarbons in presence. An infrared detection device is also included in the ROCK-EVAL® device in order to measure the quantity of non-hydrocarbon compounds (CO and CO₂) from the pyrolyzed and subsequently oxidized sample.

Thus, the ROCK-EVAL® device allows in particular to measure the quantity of hydrocarbon compounds released throughout pyrolysis. A thermogram, which is a curve showing the evolution of the quantity of a released product (hydrocarbon compounds for example) in relation to the weight of the sample considered, as a function of time, can then be generated. A thermogram generally has several peaks (see for example the peaks in FIG. 2) that are generally well differentiated. From the area of one of these peaks, a quantity representative of the quantity of product released (hydrocarbon compounds for example) in the temperature range that bounds the peak considered is obtained. Accurate information on the quantity of total organic carbon (TOC) and the quantity of mineral carbon (MinC) contained in a rock sample can also be obtained.

The method called Basic method or Bulk Rock method is also known, which can be implemented using the ROCK-EVAL® device, and which is more particularly dedicated to mother rock samples. This method is notably described in the document (Behar et al., 2001). The temperature sequence of this method is characterized by an initial temperature T1 of the pyrolysis oven, generally ranging between 300° C. and 350° C. a temperature that is maintained for a predetermined period of a few minutes. It is during this phase that the hydrocarbons referred to as “free” (actually corresponding to light to heavy molecular weight hydrocarbons), initially contained in the rock sample, are released. Their quantity is assessed by measuring the area of a first peak, denoted by S₁. The pyrolysis temperature is subsequently progressively increased, up to a temperature T2 of generally 650° C. During this phase, volatilization of the very heavy hydrocarbon compounds and cracking of the non-volatile organic matter (kerogen) occurs. The quantity of hydrocarbon compounds released during this thermal cracking phase is assessed by measuring the area of a second peak, denoted by S₂. It corresponds to the quantity of hydrocarbon compounds that would have been generated if the rock had reached a sufficient stage of maturity and burial.

The method known as Reservoir method, which can also be implemented using the ROCK-EVAL® device, is more particularly dedicated to reservoir rock and oil samples. This method is notably described in the document EP-0691540 B1 (U.S. Pat. No. 5,843,787). The temperature sequence of the Reservoir method is characterized by an initial temperature T1 of the pyrolysis oven below 200° C. and preferably equal to 180° C. This temperature is maintained for a predetermined time and the quantity of light hydrocarbon compounds is assessed by measuring the area of a first peak, denoted by S₁ᵣ. The pyrolysis oven temperature is subsequently raised to a second temperature T2 of about 370° C., a phase during which the quantity of released heavier hydrocarbons is estimated by assessing the area of a second peak, denoted by S₂ₐ. Temperature T2 substantially corresponds to the end of the thermovaporization of some hydrocarbons and to the start of pyrolysis cracking of the heavy compounds. Thus, the hydrocarbon compound family corresponding to peaks S₁ᵣ and S₂ₐ of the Reservoir method is nearly equivalent to the hydrocarbon compound family characteristic of peak S₁ of the Basic method, i.e. light to heavy molecular weight hydrocarbons. The pyrolysis temperature is subsequently raised again to a third temperature T3 of at most 650° C. The area of a third peak, denoted by S_{2b}, representative of the heavy hydrocarbon compounds, is assessed during this third heating phase. This peak S_{2b} can be considered to be equivalent to peak S₂ of the Basic method.

The method known as Shale Play, notably described in patent FR-3,021,749 (US-2015/0346179), which can also be implemented using the ROCK-EVAL® device, has been developed more recently. It is a method allowing accurate quantification of the light to heavy hydrocarbons contained in a sedimentary rock, such as a mother rock very rich in liquid hydrocarbons. This method has in particular been developed because it appeared that the Basic and Reservoir methods underestimate the area of the peak(s) corresponding to the so-called free hydrocarbons (in reality the quantity of light to heavy molecular weight hydrocarbon compounds) contained in a given rock sample. An implementation of the temperature sequence of the Shale Play method is illustrated in FIG. 1. Thus, the temperature sequence of the Shale Play method comprises a succession of three heating steps (ramps corresponding to segments A, C and E in FIG. 1) separated by two temperature maintenance steps (isothermal stages corresponding to segments B and D in FIG. 1), allowing differentiated release of the light, heavy and extra heavy hydrocarbon compounds. More precisely, the temperature sequence of the Shale Play method starts at a first low temperature (T1), ranging between 50° C. and 120° C., which allows more complete measurement of the quantity of so-called free hydrocarbon compounds (in reality of light to heavy molecular weight) present in a sample. Furthermore, the method according to the invention comprises, between two heating steps (see ramps A, C and E in FIG. 1), temperature maintenance steps (see isothermal stage B corresponding to a temperature T2 ranging between 180° C. and 220° C., and isothermal stage D corresponding to a temperature T3 ranging between 330° C. and 370° C., in FIG. 1), which allows to reach with certainty the end of the thermovaporization of the thermovaporizable hydrocarbon compounds in the temperature range considered.

FIG. 2 shows an example of a thermogram recorded during the inert-atmosphere heating sequence as described in FIG. 1. This figure shows the presence of three peaks, denoted by Sₕ₀, Sₕ₁ and Sₕ₂, representative of the quantity of hydrocarbon compounds released during the various heating steps. More precisely, peak Sₕ₀ corresponds to the quantity of hydrocarbon compounds released between first temperature T1 and second temperature T2, i.e. during segments A and B of FIG. 1. This peak Sₕ₀ is representative of the lighter hydrocarbon compounds. Peak Sₕ₁ corresponds to the quantity of hydrocarbon compounds released between second temperature T2 and third temperature T3, i.e. during segments C and D of FIG. 1. This peak Sₕ₁ is representative of the heavy thermovaporizable hydrocarbons. Peak Sₕ₂ corresponds to the quantity of hydrocarbon compounds released between third temperature T3 and fourth temperature T4, i.e. during segment E of FIG. 1. This peak Sₕ₂ is representative of the extra-heavy thermovaporizable hydrocarbons and of the cracking of non-volatile organic matter (kerogen). These methods of the applicant have been applied only to detect the liquid hydrocarbons and the potential of oil samples (for example mother rocks, reservoir rocks, oils, kerogens, among others), and to characterize them more or less accurately.

The invention aims to provide a novel method for characterization of the presence or the absence of a polymer in a porous medium and/or for quantification of this polymer in the porous medium in a simple and rapid manner.

### SUMMARY OF THE INVENTION

To this end, the invention concerns a method for characterization of the presence (or the absence) of at least one polymer in a porous medium and/or for quantification of at least this polymer in the porous medium, the porous medium being preferably a natural porous medium. In the method, at least the following steps are carried out:
a) heating a sample of the porous medium according to a first heating sequence in an inert atmosphere, and continuously measuring a representative quantity of hydrocarbon compounds, a representative quantity of carbon monoxide and/or a representative quantity of carbon dioxide released during the first heating sequence,
b) heating a sample residue from the first heating sequence according to a second heating sequence in an oxidizing atmosphere, and continuously measuring a representative quantity of carbon monoxide and/or a representative quantity of carbon dioxide released during the second heating sequence,
c) determining at least one parameter from at least one curve of the measured representative quantity of hydrocarbon compounds released during the first heating sequence and/or from a curve of the measured representative quantity of carbon monoxide released during the first heating sequence and/or from a curve of the measured representative quantity of carbon monoxide released during the second heating sequence and/or from a curve of the measured representative quantity of carbon dioxide released during the first heating sequence and/or from a curve of the measured representative quantity of carbon dioxide released during the second heating sequence, and comparing this parameter with at least one reference parameter of a first database relative to said at least one polymer, preferably with at least one reference parameter of a second database relative to at least one matrix representative of the porous medium,
d) from the comparison of the parameter determined from at least one of the curves of one of the measured quantities (i.e. the measured representative quantity of hydrocarbon compounds, carbon monoxide or carbon dioxide released during the first and/or second heating sequence) with the at least one reference parameter of a first database relative to at least the polymer and preferably with the at least one reference parameter of a second database relative to at least one matrix representative of the porous medium, characterizing the presence or the absence of the polymer (of at least one polymer) in the porous medium, and/or determining a quantity of this polymer (preferably of several polymers) in the porous medium.

Preferably, in step c), at least one temperature corresponding to a peak of the curve of the measured representative quantity of hydrocarbon compounds released during the first heating sequence and/or at least one temperature corresponding to a peak of the curve of the measured representative quantity of carbon monoxide released during the first heating sequence and/or at least one temperature corresponding to a peak of the curve of the measured representative quantity of carbon dioxide released during the first heating sequence and/or at least one temperature corresponding to a peak of the curve of the measured representative quantity of carbon monoxide released during the second heating sequence and/or at least one temperature corresponding to a peak of the curve of the measured representative quantity of carbon dioxide released during the second heating sequence is determined.

Advantageously, the first database is built as follows:
I) defining several polymer types, preferably at least polyethylene terephthalate, polyethylene, polyamide and/or perfluoroalkoxy,
II) for each defined polymer type, applying steps a) and b) to a sample of each defined polymer type in place of the porous medium sample, and determining for each defined polymer type the at least one reference parameter of the first database, the at least one reference parameter of the first database comprising at least one temperature to which the following correspond:
   a peak of the curve of the measured representative quantity of hydrocarbon compounds released by the defined polymer type sample during the first heating sequence and/or
   a peak of the curve of the measured representative quantity of carbon monoxide released by the defined polymer type sample during the first heating sequence and/or
   a peak of the curve of the measured representative quantity of carbon dioxide released by the defined polymer type sample during the first heating sequence and/or
   a peak of the curve of the measured representative quantity of carbon monoxide released by the defined polymer type sample during the second heating sequence and/or
   a peak of the curve of the measured representative quantity of carbon dioxide released by the defined polymer type sample during the second heating sequence,
      and the at least one reference parameter of the first database further comprising at least:
a representative quantity of hydrocarbon compounds released by the defined polymer type sample during the first heating sequence and/or
   a representative quantity of carbon monoxide and/or a representative quantity of carbon dioxide released by the defined polymer type sample during the first heating sequence and/or
   a representative quantity of carbon monoxide and/or a representative quantity of carbon dioxide released by the defined polymer type sample during the second heating sequence,
III) adding to the first database, for each defined polymer type, the at least one reference parameter of the first database.

Preferably, step II) is repeated with several samples of each defined polymer type.

Advantageously, in step c), at least one of the temperatures determined in step c) for the porous medium sample is compared with at least one corresponding temperature of the at least one reference parameter of said first database.

Advantageously, the second database is built as follows:
i) defining several types of porous media matrices, preferably the matrix types comprise at least sand, mar, carbonates and clays,
ii) for each defined matrix type, carrying out steps a) and b) with a sample of each defined matrix type in place of the sample of said porous medium, and determining for each defined matrix type the at least one reference parameter of the second database, the at least one reference parameter of the second database comprising at least one temperature to which the following correspond:
   a peak of said curve of said measured representative quantity of hydrocarbon compounds released by said sample of said defined matrix type during said first heating sequence and/or
   a peak of said curve of said measured representative quantity of carbon monoxide released by said sample of said defined matrix type during said first heating sequence and/or
   a peak of said curve of said measured representative quantity of carbon dioxide released by said sample of said defined matrix type during said first heating sequence and/or
   a peak of said curve of said measured representative quantity of carbon monoxide released by said sample of said defined matrix type during said second heating sequence and/or
   a peak of said curve of said measured representative quantity of carbon dioxide released by said sample of said defined matrix type during said second heating sequence,
      and the at least one reference parameter of the second database preferably further comprising at least:
a representative quantity of hydrocarbon compounds released by said sample of said defined matrix type during said first heating sequence and/or
a representative quantity of carbon monoxide and/or a representative quantity of carbon dioxide released by said sample of said defined matrix type during said first heating sequence and/or
a representative quantity of carbon monoxide and/or a representative quantity of carbon dioxide released by said sample of said defined matrix type during said second heating sequence,
iii) adding to the second database, for each defined matrix type, the reference parameter(s) of the second database.

Preferably, step ii) is repeated with several samples of each defined matrix type.

According to an implementation of the invention, in step c), at least one of the temperatures determined in step c) is compared with at least one corresponding temperature of the at least one reference parameter of said second database.

Preferably, in step c), the comparison is made by calculating at least a difference between at least one temperature corresponding to said peak of one of said curves of the quantities measured on said sample of said porous medium and the corresponding temperature of the at least one reference parameter for each defined polymer type of the first database, and in step d), if, for at least one of the defined polymer types, one at least of these differences is below a predetermined threshold, one concludes to the presence of this defined polymer type in said porous medium and, in the opposite case, one concludes to the absence of this defined polymer type in said porous medium.

According to a configuration of the invention, in step d), if one has concluded to the presence of said defined polymer type in said porous medium, said type of said defined polymer is quantified in said porous medium by determining a percentage of said defined polymer type in said porous medium from a ratio between the measured representative quantity of hydrocarbon compounds released during the first heating sequence in the porous medium sample and the measured representative quantity of hydrocarbon compounds released by said defined polymer type during the first heating sequence, and if one has concluded to the absence of said defined polymer type in said porous medium, a zero quantity is assigned to said defined polymer type.

According to an advantageous variant of the invention, in step c), the measured representative quantities of hydrocarbon compounds, carbon monoxide and carbon dioxide are normalized by the initial mass of the sample.

According to an implementation of the invention, said first heating sequence in an inert atmosphere comprises at least the following step: from a temperature ranging between 100° C. and 300° C., the temperature is raised according to a temperature gradient ranging between 5 and 30° C./minute, up to a temperature ranging between 500° C. and 650° C.

According to a variant of the invention, said second heating sequence in an oxidizing atmosphere comprises at least the following step: from a temperature ranging between 200° C. and 400° C., the temperature is raised according to a temperature gradient ranging between 10 and 40°/minute, up to a temperature ranging between 750° C. and 950° C.

### BRIEF DESCRIPTION OF THE FIGURES

Other features and advantages of the method according to the invention will be clear from reading the description hereafter of embodiments given by way of non-limitative example, with reference to the accompanying figures wherein:

FIG. 1 illustrates a variant of an inert-atmosphere heating sequence of the method according to the invention,

FIG. 2 illustrates the evolution of the quantity of hydrocarbon compounds (Q) with time (t) during a pyrolysis determined for a given sample, according to the inert-atmosphere heating sequence of FIG. 1,

FIG. 3 illustrates a measurement thermogram of one of the quantities during a heating sequence according to the invention,

FIG. 4 illustrates the evolution of the maximum temperature of the hydrocarbon compound release peak (Tpeak) during the inert-phase heating sequence in various samples of a mixture of sand and PET polymer containing different PET concentrations,

FIG. 5 illustrates the curves of hydrocarbon compound release during an inert-atmosphere heating sequence, for a PET polymer sample and for a PFA polymer sample,

FIG. 6 illustrates the curves of carbon monoxide (CO) release during an inert-atmosphere heating sequence, for a PET polymer sample and for a PFA polymer sample,

FIG. 7 illustrates the curves of carbon dioxide (CO₂) release during an inert-atmosphere heating sequence, for a PET polymer sample and for a PFA polymer sample,

FIG. 8 illustrates the curves of carbon monoxide (CO) release during an oxidizing-atmosphere heating sequence, for a PET polymer sample and for a PFA polymer sample,

FIG. 9 illustrates the curves of carbon dioxide (CO₂) release during an oxidizing-atmosphere heating sequence, for a PET polymer sample and for a PFA polymer sample,

FIG. 10a illustrates a measurement example during a heating sequence for various porous media samples, the various porous media consisting of a sand matrix and of a single PET polymer, the samples being distinguished from one another by different PET quantities in the porous medium,

FIG. 10b illustrates the evolution of the quantity of total organic carbon released, the quantity of hydrocarbon compounds released, the quantity of carbon monoxide released and the quantity of carbon dioxide released with samples of a mixture of a sand matrix and PET for various PET concentrations in the sample,

FIG. 11 illustrates relations between the quantity measured under a product release peak (hydrocarbon compounds here) as a function of the quantity of polymer present in a porous medium, for various matrix types, and

FIG. 12 illustrates an example of identification of a polymer in a real porous medium using the method according to the invention.

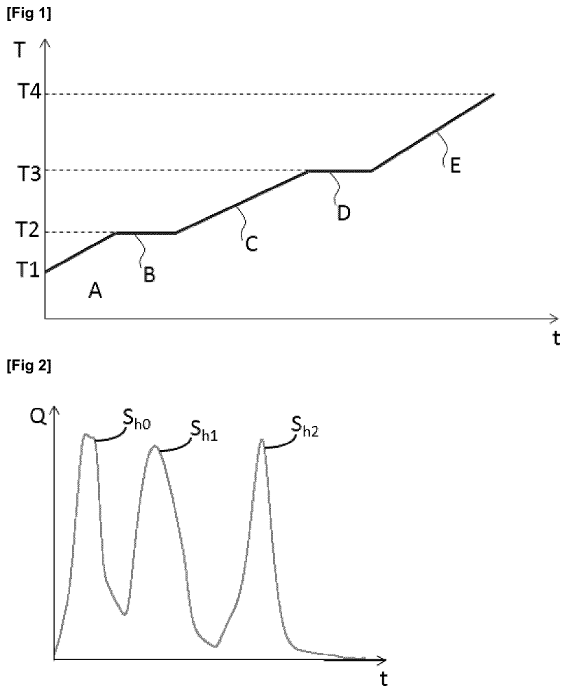

### DETAILED DESCRIPTION OF THE EMBODIMENTS

The invention concerns a method for characterization of the presence (or the absence) of at least one polymer in a porous medium, such as a natural porous medium, and/or for quantification of at least one polymer in this porous medium. The porous medium is preferably taken from a natural environment, possibly polluted by polymers, such as a beach. In this method, at least the following steps are carried out:
a) heating a sample of the porous medium according to a first heating sequence in an inert atmosphere (non-oxidizing, i.e. oxygen-free), and continuously measuring a representative quantity of hydrocarbon compounds released during the first heating sequence, a representative quantity of carbon monoxide and/or a representative quantity of carbon dioxide released during the first heating sequence. Carbon monoxide and carbon dioxide are generally representative of so-called “mineral” carbon, i.e. from carbonate materials for example. The polymers generally consist of the elements carbon and hydrogen. Thus, the measurement of hydrocarbon compounds is more easily representative of polymers. To detect the hydrocarbon compounds, this phase in an inert atmosphere is essential. Indeed, if this phase were carried out in an oxidizing atmosphere, the hydrocarbon compounds would react with the oxygen and be transformed. It would thus be difficult, or even impossible, to detect them accurately,
b) heating a sample residue from the first heating sequence according to a second heating sequence in an oxidizing atmosphere, and continuously measuring a representative quantity of carbon monoxide and/or a representative quantity of carbon dioxide released during the second heating sequence. This phase allows to detect the rest of the carbon materials undetected during the inert-atmosphere phase. It allows to refine the measurements and therefore the characterizations resulting therefrom. It also allows to determine the quantity of total organic carbon (TOC) in the samples per mass of analyzed polymer,
c) determining at least one parameter from at least one curve of the measured representative quantity of hydrocarbon compounds released during the first heating sequence and/or from a curve of the measured representative quantity of carbon monoxide released during the first heating sequence and/or from a curve of the measured representative quantity of carbon monoxide released during the second heating sequence and/or from a curve of the measured representative quantity of carbon dioxide released during the first heating sequence and/or from a curve of the measured representative quantity of carbon dioxide released during the second heating sequence, and comparing said at least one parameter with at least one reference parameter of a first database relative to said at least one polymer, and preferably with at least one reference parameter (or reference characteristic) of a second database relative to at least one matrix representative of the porous medium. Thus, the proportion of the released compounds (hydrocarbon compounds, carbon monoxide and/or carbon dioxide) from the matrix can be distinguished in these comparisons from the proportion resulting from the polymers contained in the porous medium.

Indeed, the porous medium sample that is tested and analyzed generally comprises a matrix and potentially one or more polymers that pollute the matrix. In the sense of the invention, the matrix is the “pure” porous medium, i.e. free of any polymer. The matrix can thus be clay, sand, marl, carbonates or any other pure (or clean, i.e. not polluted by polymers) natural porous medium. The first database identifies reference parameters for various polymer types, while the second database identifies reference parameters (or reference characteristics) for various matrix types. The reference parameters of the first and second database are used as a basis for comparison for the parameters determined from the measured quantity curves,
d) from the comparison of the parameter (preferably the parameters) determined from at least one of the curves of one of the measured quantities with the reference parameters of a first database relative to various polymer types (at least one polymer) and preferably with the reference parameters of a second database relative to at least one matrix representative of the porous medium, characterizing the presence or the absence of at least one polymer in the porous medium, and/or determining a quantity of this polymer in the porous medium (this polymer is quantified in the porous medium).

The presence or the absence of a polymer in the porous medium can be characterized using a characterization parameter that can comprise a binary vector, the binary values being either “zero” or “one”, “zero” corresponding for example to the absence of polymer in the porous medium, and “one” corresponding to the presence of polymer in the porous medium. The vector can thus identify, for each polymer defined in the first database, the presence or the absence of polymer in the porous medium. Thus, for example, if the first database identifies the following three polymers: polyethylene (PE), perfluoroalkoxy (PFA) and polytetrafluoroethylene (PTFE), the characterization parameter corresponding to the vector (1;0;0) identifies the presence of PE in the porous medium, and the absence of PFA and of PTFE in this porous medium.

The characterization parameter can also comprise the estimated quantity (in mass, in mass percent of the porous medium sample, or in volume percent of the porous medium sample for example) of each polymer type of the first database in the porous medium. Thus, the characterization parameter can comprise a real-valued vector. For example, for the previous case where only the PE was identified in the porous medium, the characterization parameter can comprise the following vector (2.3;0;0), in addition to or in place of the aforementioned binary vector. Thus, in this case for example, the weight of PE in the porous medium is estimated at approximately 2.3 mass %, the other values of PFA and PTFE are zero considering the identified absence of these polymers in the porous medium.

This method thus allows to assess the level of pollution of a natural porous medium by polymers, to identify the prevailing polymers in the environment of the porous medium, and this method allows to assess the evolution of the local porous medium pollution. Furthermore, the method is simple and rapid as it allows identification of the polymers in the porous medium in about 30 minutes, without prior preparation of the porous medium sample.

A porous medium is understood to be a porous medium that can be “consolidated”, i.e. consisting of a single solid block such as a rock, or “unconsolidated”, i.e. consisting of a multiplicity of solid grains, such as sand.

The porous medium can advantageously come from an underground geological formation, such as a sedimentary rock, or from a superficial geological formation forming a soil, such as a sand layer or a superficial rock.

The reference parameters of the first database relative to the polymers and of the second database relative to the porous medium matrices (these reference parameters of the second database can also be referred to as “reference characteristics” so as to differentiate them from the reference parameters of the first database) correspond to the parameters determined from a curve of at least one measured representative quantity of hydrocarbon compounds released in an inert atmosphere, of carbon monoxide and/or carbon dioxide released in an inert atmosphere and/or in an oxidizing atmosphere, respectively released by at least one polymer (for the reference parameters) and by at least one matrix (for the reference characteristics). Thus, these reference parameters and characteristics can be compared with the measurements performed on the porous medium sample in an inert atmosphere and in an oxidizing atmosphere. Thus, when one considers a reference parameter of the first or of the second database corresponding to a parameter determined in step c), it is the same parameter measured on a polymer sample (for the first database) or on a matrix sample (for the second database) in place of the porous medium sample.

For example, the various sample measurements can be compared with the same measurements performed on different polymer types.

By “same measurements”, it is either meant:
the measured representative quantities of hydrocarbon compounds released in an inert atmosphere,
the measured representative quantities of carbon monoxide released in an inert atmosphere,
the measured representative quantities of carbon dioxide released in an inert atmosphere,
the measured representative quantities of carbon monoxide released in an oxidizing atmosphere,
the measured representative quantities of carbon dioxide released in an oxidizing atmosphere.

Thus, for each porous medium sample, there are at least five different measurements (five thermograms, each thermogram showing a curve representing the measurement performed over time during the heating sequence considered). Furthermore, for each polymer of the first database and for each matrix of the second database, the reference parameters and characteristics also comprise each five reference data corresponding to the five porous medium thermograms.

Thus, the part obtained from the matrix and the part obtained from one or more polymers can be discriminated in the various thermograms generated with the porous medium sample.

### a) Inert-Atmosphere Heating Step

Advantageously, the first inert-atmosphere heating sequence can comprise at least the following step: from a temperature ranging between 100° C. and 300° C., the temperature is raised according to a temperature gradient ranging between 5 and 30° C./minute, up to a temperature ranging between 500° C. and 650° C. A linear temperature evolution is thus obtained, rapid enough to obtain fast results, and slow enough to obtain curves that can be interpreted, i.e. allowing progressive release of the hydrocarbon compounds, the carbon monoxide and the carbon dioxide as a function of the temperature evolution.

### b) Oxidizing-Atmosphere Heating Step

Preferably, the second heating sequence in an oxidizing atmosphere can comprise at least the following step: from a temperature ranging between 200° C. and 400° C. the temperature is raised according to a temperature gradient ranging between 10 and 40° C./minute, up to a temperature ranging between 750° C. and 950° C. A linear temperature evolution is thus obtained, rapid enough to obtain fast results, and slow enough to obtain curves that can be interpreted, i.e. allowing progressive release of the carbon monoxide and the carbon dioxide as a function of the temperature evolution.

Advantageously, the heating sequences of the Shale Play method described above and illustrated in FIG. 1 can be used in steps a) and b). Indeed, these heating sequences facilitate distinction of the peaks between the various polymers.

Alternatively, the heating sequences of the Basic or Reservoir methods can also be used in steps a) and b). They also allow efficient distinction between the various polymers.

c) Step of Determining at Least One Parameter and of Comparing this Parameter with the Reference Parameters of the First Database and/or of the Second Database

From the measured representative quantities of hydrocarbon compounds, CO and CO₂ in the first and second heating sequence (in an inert atmosphere and in an oxidizing atmosphere respectively), one or more parameters can be deduced from these curves, for example the temperatures corresponding to the peaks in these curves or areas under these measurement curves or in part of these curves. The measurements can preferably be normalized by the initial mass of the sample. When the measurement curves have peaks (with an ascending part upstream from the peak and a descending part downstream from the peak), a parameter can for example correspond to the area under these peaks. These parameters can be expressed, for example, in percentage of the fraction of the compounds detected by each temperature interval. Thus, the area under each peak can be distinguished. For example, when several peaks are identified on the same measurement curve (same thermogram), the area under each peak of the curve can be calculated by stopping at the troughs between the successive peaks.

To compare the various measurements, the temperature at which a product (hydrocarbon compounds, carbon monoxide or carbon dioxide) release peak occurs can notably be used. Indeed, one or more release peaks corresponding to a temperature of the heating sequence considered can be identified in each thermogram. A release peak is defined by an increasing evolution of the measurement upstream from the peak (ahead of the heating sequence considered) and by a decreasing evolution of the measurement downstream from the peak (toward the end of the heating sequence considered), in the neighborhood of the peak. A peak thus corresponds to a local maximum of the measurement curve (of the thermogram).

Advantageously, in step c), the measured quantities of hydrocarbon compounds, carbon monoxide and carbon dioxide can be normalized by the initial mass of the porous medium sample. By normalization, it is meant that the considered quantity (of hydrocarbon compounds, carbon monoxide or carbon dioxide during the first or the second heating sequence) is divided by the initial mass of the sample considered, the initial mass being the mass before the pyrolysis a) and oxidation b) steps.

Furthermore, in the sense of the invention, all the aforementioned quantities are advantageously normalized in relation to the relative sample weight (porous medium, polymer or matrix). Thus, comparing these various measurements is facilitated through normalization.

Advantageously, the reference parameter (preferably the reference parameters) of the first database can comprise, for several polymer types, a temperature to which the following correspond:
a peak of the curve of the measured representative quantity of hydrocarbon compounds released by the defined polymer type sample during the first heating sequence and/or
a peak of the curve of the measured representative quantity of carbon monoxide released by the defined polymer type sample during the first heating sequence and/or
a peak of the curve of the measured representative quantity of carbon dioxide released by the defined polymer type sample during the first heating sequence and/or
a peak of the curve of the measured representative quantity of carbon monoxide released by the defined polymer type sample during the second heating sequence and/or
a peak of the curve of the measured representative quantity of carbon dioxide released by the defined polymer type sample during the second heating sequence,
   and the reference parameter of the first database preferably further comprising at least:
a representative quantity of hydrocarbon compounds released by the defined polymer type sample during the first heating sequence and/or
a representative quantity of carbon monoxide and/or a representative quantity of carbon dioxide released by the defined polymer type sample during the first heating sequence and/or
a representative quantity of carbon monoxide and/or a representative quantity of carbon dioxide released by the defined polymer type sample during the second heating sequence.

The quantities and temperatures defined above are understood for each polymer type alone. In other words, these are the quantities and temperatures that would be obtained with a porous medium sample only consisting of the polymer considered (the sample would be a sample of the polymer considered alone as “pure”).

Thus, for each polymer considered in the first database, the quantities and temperatures listed above, which correspond to the reference parameters of the polymer considered and provide the identity of each polymer, are identified.

When comparing the measurements performed on the porous medium sample with the reference parameters of the various polymers listed above, it goes without saying that the corresponding quantities are compared: for example, the representative quantity of hydrocarbon compounds released by the porous medium sample during said first heating sequence is compared with the same quantity (the representative quantity of hydrocarbon compounds during said first sequence) released by the polymer considered. Thus, in the sense of the invention, it would be meaningless to compare for example the representative quantity of hydrocarbon compounds released by the porous medium sample during the first heating sequence with the representative quantity of carbon monoxide released by the polymer considered during the first sequence.

Advantageously, in a similar manner, the reference parameter (preferably the reference parameters) of the second database can comprise, for several matrices, at least one temperature to which the following correspond:
a peak of the curve of the measured representative quantity of hydrocarbon compounds released by the defined matrix type sample during the first heating sequence and/or
a peak of the curve of the measured representative quantity of carbon monoxide released by the defined matrix type sample during the first heating sequence and/or
a peak of the curve of the measured representative quantity of carbon dioxide released by the defined matrix type sample during the first heating sequence and/or
a peak of the curve of the measured representative quantity of carbon monoxide released by the defined matrix type sample during the second heating sequence and/or
a peak of the curve of the measured representative quantity of carbon dioxide released by the defined matrix type sample during the second heating sequence,
   and the reference parameter of the second database preferably further comprising at least:
a representative quantity of hydrocarbon compounds released by the defined matrix type sample during the first heating sequence and/or
a representative quantity of carbon monoxide and/or a representative quantity of carbon dioxide released by the defined matrix type sample during the first heating sequence and/or
a representative quantity of carbon monoxide and/or a representative quantity of carbon dioxide released by the defined matrix type sample during the second heating sequence.

The quantities and temperatures defined above are understood for the pure matrix alone, i.e. not polluted by any polymer. In other words, these are the quantities and temperatures that would be obtained with a porous medium sample only consisting of the matrix considered, without polymers (the sample would be a sample of the matrix alone considered as “pure”).

Thus, for each matrix considered in the second database, the quantities and temperatures listed above, which correspond to the reference characteristics of the matrix considered and provide the identity of each matrix, are identified.

When comparing the measurements performed on the porous medium sample with the reference parameters of the various matrices of the second database listed above, it goes without saying that the corresponding quantities are compared: for example, the representative quantity of hydrocarbon compounds released by the porous medium sample during the first heating sequence is compared with the same quantity (the representative quantity of hydrocarbon compounds during said first sequence) released by the matrix considered. Thus, in the sense of the invention, it would be meaningless to compare for example the representative quantity of hydrocarbon compounds released by the porous medium sample during the first heating sequence with the representative quantity of carbon monoxide released by the matrix considered during the first sequence.

According to a preferred implementation of the invention, the first database can be built as follows:
I) defining several polymer types preferably, at least polyethylene terephthalate (PET), polyethylene (PE), polyamide (PA), perfluoroalkoxy (PFA) and/or polypropylene (PP).

Preferably, in order to better target the polymers found in the porous medium, several grades of each polymer can be used, for example several polyethylene or polyamide grades,
II) for each defined polymer type, applying steps a) and b) of the method (i.e. the two heating sequences simultaneously with the planned measurements) to a sample of each defined polymer type in place of the porous medium sample, and determining at least one reference parameter (preferably several reference parameters) of the first database for each defined polymer type. A defined polymer sample is understood to be a polymer sample alone (or pure), i.e. without matrix. In other words, the polymer makes up 100% of the porous medium,
III) adding to the first database, for each defined polymer type, the reference parameter(s) of the first database.

From the five thermograms obtained with the sample of each defined polymer (pure, without matrix), the five thermograms being the measurement curves of the measured representative quantities of hydrocarbon compounds during the first heating sequence, and of the measured representative quantities of carbon monoxide and carbon dioxide during the first and second heating sequences, the following normalized different quantities can be calculated:

$$\begin{matrix}Total {HC}_{polymer} = [ FID {signal}_{polymer} / Initial {mass}_{polymer} ] \\ Total {CO}_{polymer} = [ IR CO {signal}_{polymer} / Initial {mass}_{polymer} ] \\ Total {CO}_{2 polymer} = [ IR {CO}_{2} {signal}_{polymer} / Initial {mass}_{polymer} ]\end{matrix}$$

with:
Total HC_{polymer} corresponding to the quantity of total hydrocarbon compounds released by the polymer sample (alone, without matrix) during the inert-atmosphere phase,
Total CO_{polymer} corresponding to the quantity of total carbon monoxide released by the polymer sample (alone, without matrix), corresponding to the sum of the quantity of carbon monoxide released by the polymer sample (alone, without matrix) during the inert-atmosphere phase and during the oxidizing phase,
Total CO_{2 polymer} corresponding to the quantity of total carbon dioxide released by the polymer sample (alone, without matrix), corresponding to the sum of the quantity of carbon dioxide released by the polymer sample (alone, without matrix) during the inert-atmosphere phase and during the oxidizing phase,
FID signal_{polymer} corresponding to the area under the signal measured by the FID (flame ionization) detector during the inert-atmosphere phase with the polymer sample (pure, without matrix),
IR CO signal_{polymer} corresponding to the area under the signal measured by the infrared detector measuring the carbon monoxide CO during the inert-atmosphere phase and during the oxidizing-atmosphere phase, thus the sum of the areas under the signals of these two phases, with the polymer sample (pure, without matrix),
IR CO₂ signal_{polymer} corresponding to the area under the signal measured by the infrared detector measuring the carbon dioxide CO₂ during the inert-atmosphere phase and during the oxidizing-atmosphere phase, thus the sum of the areas under the signals of these two phases, with the polymer sample (pure, without matrix),
Initial mass polymer corresponding to the initial mass of the polymer sample (alone, without matrix).

Furthermore, at least the temperature Tpeak_{HCpolymer} of the hydrocarbon compound release peak during the first heating sequence can be identified from the defined polymer sample alone (or pure):

$${Tpeak}_{HCpolymer} = [ Tamperature of FID signal {maximum}_{HCpolymer} ] ( ° C . )$$

where: Temperature of FID signal maximum_{HCpolymer} is the temperature corresponding to the maximum measurement of the curve measured by the FID (flame ionization) detector, therefore the hydrocarbon compound release peak.

The following can also be identified:
the temperature of the carbon monoxide release peak during the first heating sequence from the defined polymer sample alone (or pure):

$${Tpeak}_{COpolymer \_ S 1} = [ Tamperature of IR signal {maximum}_{COpolymer \_ S 1} ] ( ° C . )$$

where: Temperature of IR signal maximum_{COpolymer_S1} is the temperature corresponding to the maximum measurement of the curve measured by the infrared carbon monoxide detector, therefore the carbon monoxide release peak during the first heating sequence,
the temperature of the carbon dioxide release peak during the first heating sequence from the defined polymer sample alone (or pure):

$${Tpeak}_{CO 2 polymer \_ S 1} = [ Tamperature of IR signal {maximum}_{CO 2 polymer \_ S 1} ] ( ° C . )$$

where: Temperature of IR signal maximum_{CO2polymer_S1} is the temperature corresponding to the maximum measurement of the curve measured by the infrared carbon dioxide detector, therefore the carbon dioxide release peak during the first heating sequence,
the temperature of the carbon monoxide release peak during the second heating sequence from the defined polymer sample alone (or pure):

$${Tpeak}_{COpolymer \_ S 2} = [ Tamperature of IR signal {maximum}_{Copolymer \_ S 2} ] ( ° C . )$$

where: Temperature of IR signal maximum_{COpolymer_S2} is the temperature corresponding to the maximum measurement of the curve measured by the infrared carbon monoxide detector, therefore the carbon monoxide release peak during the second heating sequence,
the temperature of the carbon dioxide release peak during the second heating sequence from the defined polymer sample alone (or pure):

$${Tpeak}_{CO 2 polymer \_ S 2} = [ Temperature of IR signal {maximum}_{CO 2 polymer \_ S 2} ] ( ° C . )$$

where: Temperature of IR signal maximum_{CO2polymer_S2} is the temperature corresponding to the maximum measurement of the curve measured by the infrared carbon dioxide detector, therefore the carbon dioxide release peak during the second heating sequence.

Preferably, step II) can be repeated with several samples (at least 2 and preferably between 5 and 10) of each defined polymer type. By carrying out these operations for several identical samples, the reference parameters can be assessed through the average of the values obtained with the various samples, and the accuracy of the reference parameters can be determined by defining the standard deviation.

According to a configuration of the invention, the second database can be built as follows:
i) defining several types of porous medium matrices, preferably the matrix types comprise at least sand, marl, carbonates and clays, which are the main natural matrix types,
ii) for each defined matrix type, carrying out steps a) and b) of the method (i.e. the two inert-atmosphere and oxidizing-atmosphere heating sequences simultaneously with the planned measurements) with a sample of each defined matrix type in place of the porous medium sample, and determining for each defined matrix type at least one reference parameter of the second database,
iii) adding to the second database, for each defined matrix type, the reference parameter(s) of the second database.

Preferably, step ii) can be repeated with several samples (at least 2 and preferably between 5 and 10) of each defined matrix type. By carrying out these operations for several identical samples, the reference characteristics can be assessed through the average of the values obtained with the various samples, and the accuracy of the reference characteristics can be determined by defining the standard deviation.

From the five thermograms obtained with the sample of each defined matrix, the five thermograms being the measurement curves of the representative quantities of hydrocarbon compounds during the first heating sequence, and of the representative quantities of carbon monoxide and carbon dioxide during the first and second heating sequences, the following normalized different quantities can be calculated:

$$Total {HC}_{matrix} = [ FID {signal}_{matrix} / Initial {mass}_{matrix} ] \\ Total {CO}_{matrix} = [ IR CO {signal}_{matrix} / Initial {mass}_{matrix} ] \\ Total {CO}_{2 matrix} = [ IR {CO}_{2} {signal}_{matrix} / Initial {mass}_{matrix} ]$$

with:
Total HCₘₐₜᵣᵢₓ corresponding to the quantity of total hydrocarbon compounds released by the matrix sample (alone, without polymer, i.e. “pure” or not polluted) during the inert-atmosphere phase,
Total COₘₐₜᵣᵢₓ corresponding to the quantity of total carbon monoxide released by the matrix sample (alone, without polymer, i.e. “pure” or not polluted), corresponding to the sum of the quantity of carbon monoxide released by the matrix sample (alone, without polymer, i.e. “pure” or not polluted) during the inert-atmosphere phase and during the oxidizing-atmosphere phase,
Total CO_{2 matrix} corresponding to the quantity of total carbon dioxide released by the matrix sample (alone, without polymer, i.e. “pure” or not polluted), corresponding to the sum of the quantity of carbon dioxide released by the matrix sample (alone, without polymer, i.e. “pure” or not polluted) during the inert-atmosphere phase and during the oxidizing-atmosphere phase,
FID signalₘₐₜᵣᵢₓ corresponding to the area under the signal measured by the FID (flame ionization) detector during the inert-atmosphere phase with the matrix sample (alone, without polymer, i.e. “pure” or not polluted).
IR CO signalₘₐₜᵣᵢₓ corresponding to the area under the signal measured by the infrared detector measuring the carbon monoxide CO during the inert-atmosphere phase and during the oxidizing-atmosphere phase, thus the sum of the areas under the signals of these two phases, with the matrix sample (alone, without polymer, i.e. “pure” or not polluted).
IR CO₂ signalₘₐₜᵣᵢₓ corresponding to the area under the signal measured by the infrared detector measuring the carbon dioxide CO₂ during the inert-atmosphere phase and during the oxidizing-atmosphere phase, thus the sum of the areas under the signals of these two phases, with the matrix sample (alone, without polymer, i.e. “pure” or not polluted).
Initial massₘₐₜᵣᵢₓ corresponding to the initial mass of the matrix sample (alone, without polymer, i.e. “pure” or not polluted).

Furthermore, at least the temperature Tpeak_{HCmatrix} of the hydrocarbon compound release peak during the first heating sequence can be identified from the matrix sample (alone, without polymer, i.e. “pure” or not polluted):

$${Tpeak}_{HCmatrix} = [ Temperature of FID signal {maximum}_{HCmatrix} ] ( ° C . )$$

where Temperature of FID signal maximum_{HCmatrix} is the temperature corresponding to the maximum measurement of the curve measured by the FID (flame ionization) detector, therefore the hydrocarbon compound release peak, with the matrix sample (alone, without polymer, i.e. “pure” or not polluted).

The following can also be identified:
the temperature of the carbon monoxide release peak during the first heating sequence from the matrix sample defined alone (or pure):

$${Tpeak}_{CO matrix \_ S 1} = [ Temperature of IR signal {maximum}_{CO matrix \_ S 1} ] ( ° C . )$$

where: Temperature of IR signal maximum_{COmatrice_S1} is the temperature corresponding to the maximum measurement of the curve measured by the infrared carbon monoxide detector, therefore the carbon monoxide release peak during the first heating sequence, with the matrix sample (alone, without polymer, i.e. “pure” or not polluted).
the temperature of the carbon dioxide release peak during the first heating sequence, from the matrix sample defined alone (or pure):

$${Tpeak}_{CO matrixe \_ S 1} = [ Temperature of IR signal {maximum}_{CO matrix \_ S 1} ] ( ° C . )$$

where: Temperature of IR signal maximum_{CO2matrix_S1} is the temperature corresponding to the maximum measurement of the curve measured by the infrared carbon dioxide detector, therefore the carbon dioxide release peak during the first heating sequence, with the matrix sample (alone, without polymer, i.e. “pure” or not polluted).
the temperature of the carbon monoxide release peak during the second heating sequence from the matrix sample defined alone (or pure):

$${Tpeak}_{CO matrix \_ S 2} = [ Temperature of IR signal {maximum}_{CO matrix \_ S 2} ] ( ° C . )$$

where: Temperature of IR signal maximum_{COmatrix_S2} is the temperature corresponding to the maximum measurement of the curve measured by the infrared carbon monoxide detector, therefore the carbon monoxide release peak during the second heating sequence, with the matrix sample (alone, without polymer, i.e. “pure” or not polluted).
the temperature of the carbon dioxide release peak during the second heating sequence, from the matrix sample defined alone (or pure):

$${Tpeak}_{CO 2 matrix \_ S 2} = [ Temperature of IR signal {maximum}_{CO 2 matrix \_ S 2} ] ( ° C . )$$

where: Temperature of IR signal maximum_{CO2matrix_S2} is the temperature corresponding to the maximum measurement of the curve measured by the infrared carbon dioxide detector, therefore the carbon dioxide release peak during the second heating sequence, with the matrix sample (alone, without polymer, i.e. “pure” or not polluted).

Preferably, the first and second database constructions described above can be achieved prior to step a), step b) or step c).

According to a preferred configuration of the invention, in step c), the comparison can be performed by calculating at least a difference between a temperature corresponding to the peak of a curve (or several temperatures corresponding to the peak of several curves) of the quantities measured on the porous medium sample and the corresponding temperature of a reference parameter for each defined polymer type of the first database.

The corresponding temperature or measured quantity of a reference parameter is understood to be the temperature from the same measurement curve type. For example, if the parameter of the determined porous medium is the peak temperature of a measurement curve of the representative quantity of hydrocarbon compounds during the first heating sequence, the corresponding temperature is that of the peak of a measurement curve of the representative quantity of hydrocarbon compounds during the first heating sequence obtained from the various defined polymer types of the first database or the various defined matrix types of the second database.

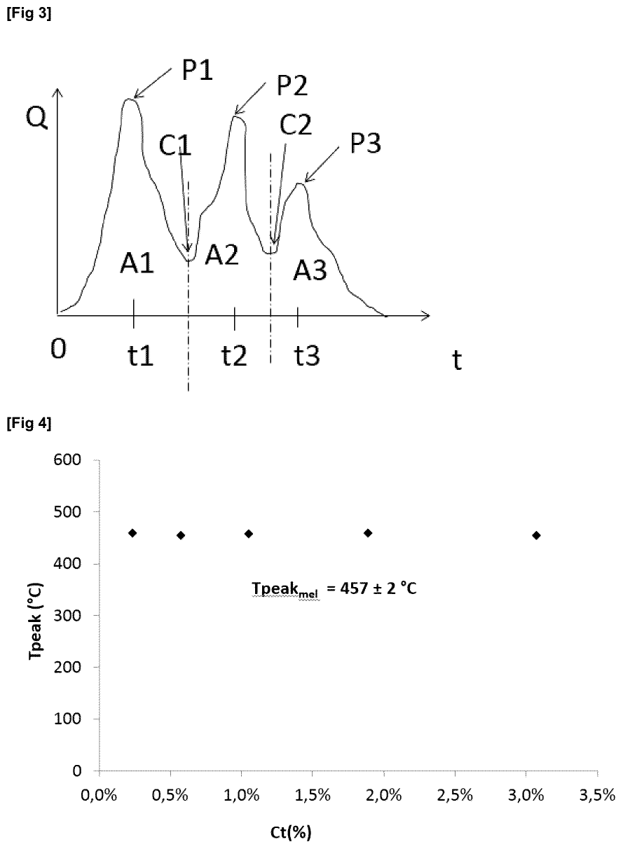

FIG. 3 schematically illustrates, by way of non-limitative example, an example of a thermogram showing a quantity Q measured over time t during a heating sequence. Quantity Q can correspond to a representative quantity of hydrocarbon compounds, of carbon monoxide or of carbon dioxide, and the heating sequence can be the first or the second heating sequence. The thermogram comprises three peak measurements P1, P2 and P3. In the neighborhood of each of these peaks P1, P2 and P3, upstream (i.e. to the left) from each of these peaks P1, P2 and P3, the curve is ascending, and downstream (i.e. to the right), the curve is descending.

The peaks being separated by troughs C1 and C2 for which, in the neighborhood of these troughs, the curve is descending before the trough (to the left of the trough) and the curve is ascending after the trough (to the right of the trough).

In the thermogram, each peak is associated with a time t1 for peak P1, 12 for peak P2 and 13 for peak P3. Now, each time of the heating sequence corresponds to a specific temperature. Thus, a specific peak temperature corresponds to each peak of the thermogram.

These different peaks P1 to P3 can correspond to the matrix and/or to one or more polymers.

To discriminate the various peaks and to assign them to a specific matrix or polymer, it is possible to compare the temperature to which each peak P1 to P3 corresponds and the peak temperatures of the quantity of reference parameters for the various polymers of the first database and of reference characteristics of the various matrices of the second database. Thus, when the temperature of a peak P1 to P3 corresponds to that of a matrix or of a polymer in a certain tolerance range (for example +/−10° C., preferably +/−5° C.), this peak can be assigned to this matrix or to this polymer.

For example, peak P1 can correspond to the marl matrix, peak P2 to a first polymer (PET for example), and peak P3 to a second polymer (PFA for example). Thus, that which comes from the matrix and that which comes from the polymers it contains can be distinguished in the thermogram of the porous medium sample. Furthermore, these polymers can be identified.

Indeed, experiments carried out by the applicant have shown that the various polymers can be distinguished by different peak temperatures characteristic of each polymer. Similarly, each matrix type is identifiable by the temperature of the peak in the thermogram. For this distinction, the hydrocarbon compound measurement thermogram is preferably used. The other thermograms can be used to confirm this first identification or to refine it: one or more of these other thermograms can be used.

To quantify the quantity of each polymer in the porous medium, the part of the measurement associated with each peak can be identified. For example, the surface area (the area) under the curve of each peak can be calculated. It is thus possible to distinguish the first area A1 that ends at first trough C1, this area being associated with the marl of the porous medium (first peak P1 being assigned to the marl type matrix), the second area A2 under the curve between first trough C1 and second trough C2, this area A2 being associated with polymer PET, and the third area A3 from second trough C3, this area A3 being associated with polymer PFA.

Of course, there are other possible means of distinguishing the quantities from the various polymers and the matrix. Different signal processings are therefore possible.

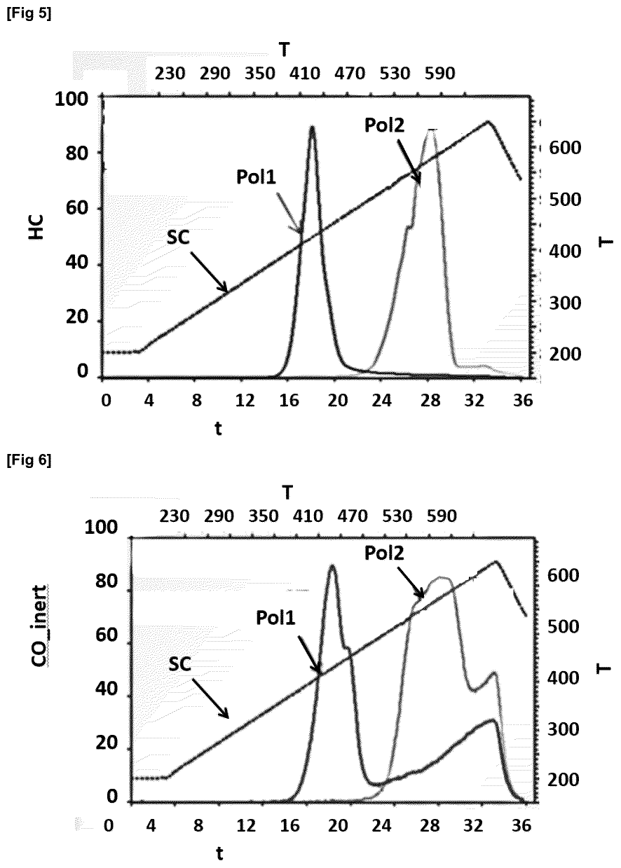

FIG. 4 schematically illustrates, by way of non-limitative example, an example carried out by the applicant. In this example, different samples of a mixture of a sand matrix (same sand in all the samples) and a (single) PET polymer are mixed together. Each sample has a different PET content Ct, the content being measured in percent by mass of PET in the sample. Thus, the five samples respectively have a PET content in the mixture of 0.2%, 0.6%, 1.1%, 1.9% and 3.1%. For each sample, FIG. 4 shows the temperature Tpeak of the hydrocarbon compound release peak measured during the inert phase as a function of the PET content of the sample considered. The curve of the measured representative hydrocarbon compound quantities in the inert phase comprises a single peak for all the samples. The various diagrams represent the peak temperature values Tpeak obtained for each sample. Thus, it has been verified that this temperature Tpeak is independent of the PET content of the sample, the temperature Tpeakₘₑₗ of the PET being constant at 457° C.+/−2° C. Tpeak thus is a robust parameter that can be used for characterization and identification of each polymer family.

Preferably, in step c), one can determine as a parameter, from at least one curve of a quantity measured in steps a) and b), at least one temperature corresponding to a peak of the curve of said measured representative quantity of hydrocarbon compounds released during the first heating sequence and/or at least one temperature corresponding to a peak of the curve of the measured representative quantity of carbon monoxide released during the first heating sequence and/or at least one temperature corresponding to a peak of the curve of the measured representative quantity of carbon dioxide released during the first heating sequences and/or at least one temperature corresponding to a peak of the curve of the measured representative quantity of carbon monoxide released during the second heating sequence and/or at least one temperature corresponding to a peak of the curve of the measured representative quantity of carbon dioxide released during the second heating sequence.

Advantageously, in step c), at least one of these temperatures can be compared with at least one corresponding temperature of the reference parameters of the first database and preferably of those of the second database (reference characteristics). Comparing this at least one temperature with the corresponding temperature of a polymer of the first database and possibly with the corresponding temperature of a matrix of the second database makes it possible to identify one or more polymers and/or the matrix of the porous medium.

A “corresponding” measurement or temperature is understood to be the same type of measurement or of temperature between those performed or obtained in steps a) and/or b) on the porous medium sample and those of the reference parameters of the first and second database for the various polymer and matrix types of these databases. These corresponding measurements and/or temperatures are those allowing proper comparisons between the porous medium sample and the polymers of the first database and the matrices of the second database. For example, if the parameter determined in step c) is the temperature of the peak obtained from the curve of the measured quantity of hydrocarbon compounds during the first heating sequence on the porous medium sample, the reference parameters of the first and second databases can then be the temperatures of the peak obtained from the curve of the measured quantity of hydrocarbon compounds during the first heating sequence on the sample of each defined polymer type of the first database or of each defined matrix type of the second database.

From the thermograms obtained on the porous medium sample (natural for example), the following different normalized quantities can also be calculated:

$$Total {HC}_{milieu poreux} = [ FID {signal}_{milieu poreux} / Initial {mass}_{milieu poreux} ] \\ Total {CO}_{milieu poreux} = [ IR CO {signal}_{milieu poreux} / Initial {mass}_{milieu poreux} ] \\ Total {CO}_{2 milieu poreux} = [ IR {CO}_{2} {signal}_{milieu poreux} / Initial {mass}_{milieu poreux} ]$$

with:
Total HC_{milieu poreux} corresponding to the quantity of total hydrocarbon compounds released by the porous medium sample during the inert-atmosphere phase,
Total CO_{milieu poreux} corresponding to the quantity of total carbon monoxide released by the porous medium sample, corresponding to the sum of the quantity of carbon monoxide released by the porous medium sample during the inert-atmosphere phase and during the oxidizing-atmosphere phase,
Total CO_{2 milieu poreux} corresponding to the quantity of total carbon dioxide released by the porous medium sample, corresponding to the sum of the quantity of carbon dioxide released by the porous medium sample during the inert-atmosphere phase and during the oxidizing-atmosphere phase,
FID signal_{milieu poreux} corresponding to the area under the signal measured by the FID (flame ionization) detector during the inert-atmosphere phase with the porous medium sample,
IR CO signal_{milieu poreux} corresponding to the area under the signal measured by the infrared detector measuring the carbon monoxide CO during the inert-atmosphere phase and during the oxidizing-atmosphere phase, thus the sum of the areas under the signals of these two phases, with the porous medium sample,
IR CO₂ signal_{milieu poreux} corresponding to the area under the signal measured by the infrared detector measuring the carbon dioxide CO₂ during the inert-atmosphere phase and during the oxidizing-atmosphere phase, thus the sum of the areas under the signals of these two phases, with the porous medium sample,
Initial mass_{milieu poreux} corresponding to the initial mass of the porous medium sample.
   d) Step of Characterization of the Presence or the Absence of Polymer in the Porous Medium and/or Quantification Thereof

The temperatures of the hydrocarbon compound release peaks during the first heating sequence can be sufficient to identify one or more polymers in the porous medium. For example, if a peak is detected at a certain temperature corresponding to a reference parameter of a specific polymer, the parameter in question being the temperature of the hydrocarbon compound release peak from a sample of polymer alone (or pure, without matrix) during the first heating sequence, the presence (or the absence) of this particular polymer can be identified (characterized). What is meant by “the temperature corresponds” is a difference less than a predefined criterion (20° C. for example, preferably 10° C.) in absolute value. For example, when the difference with the temperature of the parameter is that of a reference parameter ranging between −20° C. and +20° C. these two temperatures can be considered to correspond. In other words, when for at least the thermogram representative of hydrocarbon compounds generated with the porous medium sample, the peak temperature (to within the predefined criterion, i.e. for example +/−20° C., preferably +/−10° C.) corresponding to the thermogram representative of the hydrocarbon compounds corresponding to a particular polymer of the first database is found, it can be considered that this particular polymer is present in the porous medium.

Of course, several other temperatures of peaks of the other thermograms can also be used to refine or to confirm the identification.

When for the five thermograms generated with the porous medium sample, all the peak temperatures (to within the predefined criterion, for example +/−10° C.) corresponding to the corresponding thermograms of a particular polymer of the first database are found, the presence of this polymer in the porous medium can be confirmed, and the quantity evaluation of this polymer in the porous medium can be more accurate. Of course, this method can allow to detect in the same manner several polymers in the porous medium.

When the matrix of the porous medium is sand, the sand mainly consisting of silica generating no hydrocarbon compound and no carbon monoxide or dioxide, whether in an inert or an oxidizing atmosphere, it is not necessary to use a second database representative of different porous medium matrices. Indeed, if a sand sample is taken from a beach, the matrix of this porous medium (the pure sand, i.e. not polluted by polymers) will have thermograms without peaks. Thus, the peaks identified on the polluted sand sample should directly correspond to polymers. When the porous medium matrix can comprise carbon compounds likely to generate hydrocarbon compounds, carbon monoxide and/or carbon dioxide, using a second database representative of the matrix can be useful. When, for at least the thermogram representative of hydrocarbon compounds generated on the porous medium sample, the peak temperature (to within a predefined criterion, for example +/−20° C., preferably +/−10° C.) corresponding to the thermogram representative of hydrocarbon compounds corresponding to a particular matrix of the second database is found, it can be considered that the porous medium matrix corresponds to this particular matrix. This allows to identify, among the identified peaks of the porous medium, those corresponding to the matrix and those corresponding to one or more polymers. The other thermograms of the other measured quantities can be compared in the same manner to refine the characterization. Besides, other peaks may correspond to neither a matrix of the second database, nor to a polymer of the first database. In this case, these peaks may correspond to either an unidentified matrix in the second database, or an unidentified polymer in the first database, or a neoformed compound, i.e. a material generated by chemical reactions due to the presence of a polymer in the matrix.

According to a preferred configuration of the invention wherein, in step c), the comparison can be made by calculating at least a difference between a temperature corresponding to the peak of a curve (or several peak temperatures of several curves) of the quantities measured on the porous medium sample and the corresponding temperature of a reference parameter for each defined polymer type of the first database, in step d), if, for at least one of the defined polymer types, at least one of these differences (the absolute value of the difference) is less than a predetermined threshold (for example less than 20° C., preferably less than 10° C.), one can conclude to the presence of this defined polymer type in the porous medium and, in the opposite case, one can conclude to the absence of this defined polymer type in the porous medium. Of course, when the measurement curve comprises several peaks, several polymer types can be similarly identified from the temperatures of these different peaks.

Thus, when among the determined temperatures corresponding to a peak of a quantity of hydrocarbon compounds, carbon monoxide and carbon dioxide measured during the first and second heating sequences from the porous medium sample, it is possible to find for a polymer of the first database, to within the predetermined threshold, at least the temperature corresponding to a peak of the hydrocarbon compound quantity and preferably at least another one of the temperatures corresponding to a peak of a hydrocarbon quantity and preferably at least another one of the temperatures corresponding to a release peak for the carbon monoxide, carbon dioxide released by this polymer during the first and/or second heating sequence, the presence of this polymer in said porous medium is characterized. The presence of the various peaks located on the same temperatures of the different measurements (representative quantities of hydrocarbon compounds, carbon monoxide or carbon dioxide) of the heating sequences allows the presence of the polymer in the porous medium to be characterized and/or confirmed. Of course, several polymers can be similarly identified in the porous medium.

According to a variant of the invention, in step d), if one has concluded to the presence of a defined polymer type in the porous medium, the defined polymer type in the porous medium can be quantified (the quantity of this defined polymer in the porous medium is determined) by determining a percentage of the defined polymer type in the porous medium from a ratio between the measured representative quantity of hydrocarbon compounds released during the first heating sequence in the porous medium sample and the measured representative quantity of hydrocarbon compounds released by the defined polymer type during the first heating sequence, and if one has concluded to the absence of the defined polymer type in the porous medium, a zero quantity can be assigned to the defined polymer type.

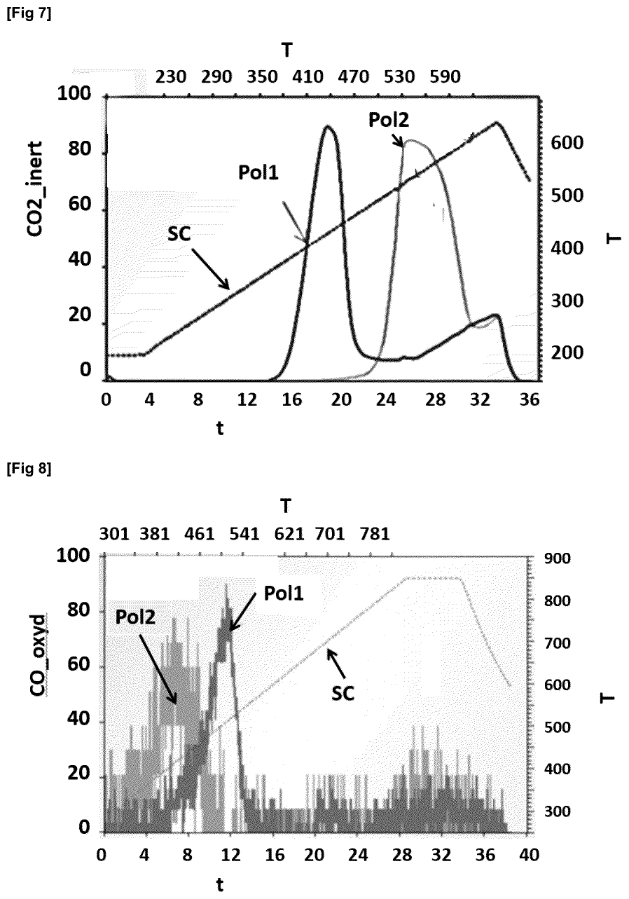

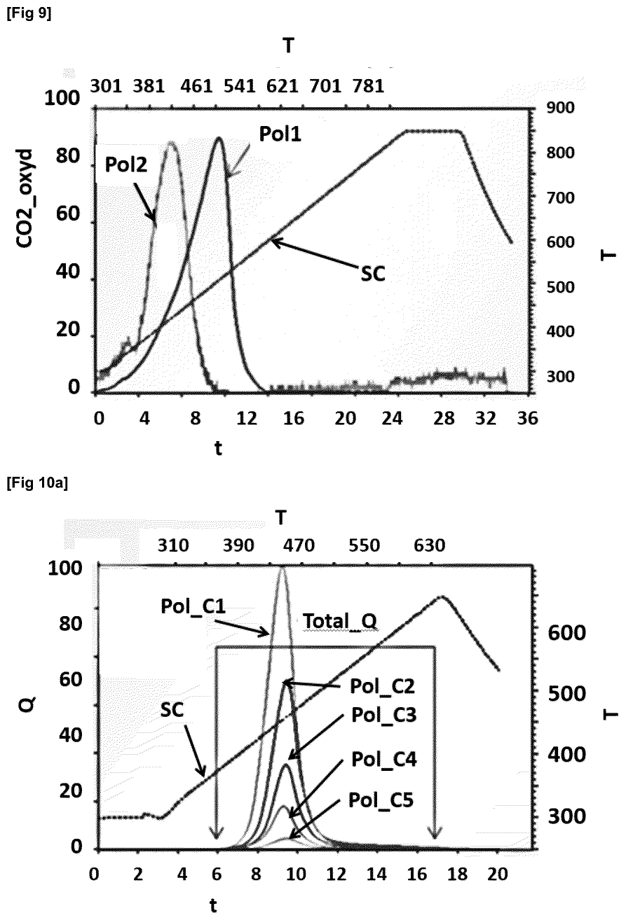

Furthermore, FIGS. 5 to 9 schematically illustrate, by way of non-limitative example, the measured quantities (respectively the quantity of hydrocarbon compounds HC during the inert phase, the quantity of carbon monoxide during the inert phase CO_inert, the quantity of carbon dioxide during the inert phase CO2_inert, the quantity of carbon monoxide during the oxidizing phase CO_oxyd and the quantity of carbon dioxide during the oxidizing phase CO2_oxyd) released over time t in minutes, during a heating sequence SC where the temperature T (in ° C.) varies over time t for a first sample Pol1 of pure PET (without matrix) and for a second sample Pol2 of pure PFA (without matrix).

FIGS. 5 to 9 are shown only by way of illustration, and independently of each other.

The heating sequences SC of one figure are identical for the two samples Pol1 and Pol2.

The quantities are given in percentage of total release during the phase considered.

These figures allow to observe that the peaks, and notably the corresponding temperatures at the time of these peaks, are different between the two samples Pol1 and Pol2. Thus, from the peaks of these different figures, a peak temperature can be identified for PET and a peak temperature can be identified for PFA in each of these figures. One or the other of the measured quantity release temperatures can thus be used to distinguish the polymer type found in a porous medium sample. The test carried out allows to distinguish PET and PFA, but the applicant has observed that the temperature of the peaks allows the polymers to be distinguished more broadly than in this example. It is noted that the peak temperature of hydrocarbon compound release is generally preferred to distinguish the polymers (because the curves of FIG. 5 are cleaner and the peaks are sharper, the width of the curve around the peaks being smaller) and that it can be sufficient. It can be noted that, for this example, the measurement curves of carbon monoxide in the oxidizing phase CO_oxyd of FIG. 8 are much less clean and that these curves are therefore less favorable to distinguish the various polymers.

Besides, the temperature of the peaks is a precise parameter with little variation, which makes it possible to distinguish the polymers more readily.

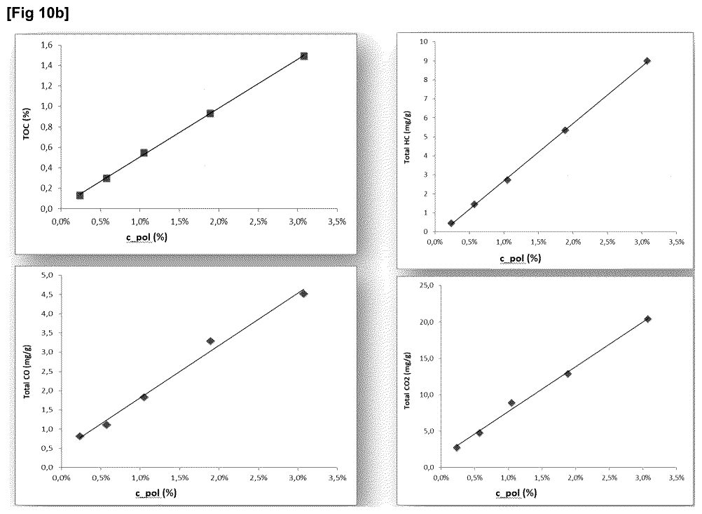

FIG. 10a schematically illustrates, by way of non-limitative example, different measurements of a quantity Q (here a quantity representative of the hydrocarbon compounds during the inert phase, but the other quantities described in the present description could be measured alternatively) over time t (in minutes) during a heating sequence SC during which temperature T evolves.

In this figure, parameter Total_Q represents the area measured under each curve of each sample during the length of time materialized between the vertical arrows connected by a horizontal segment, a time during which a release of quantity Q is observed.

The various samples Pol_C1, Pol_C2, Pol_C3, Pol_C4 and Pol_C5 were prepared from a sand matrix to which a PET polymer was added (only polymer added to the sand). The various samples Pol_C1, Pol_C2, Pol_C3, Pol_C4 and Pol_C5 are distinguished by different polymer concentrations in the porous medium. These samples Pol_C1, Pol_C2, Pol_C3, Pol_C4 and Pol_C5 are artificial, and their purpose is to check the polymer quantification in a porous medium. The polymer concentration of sample Pol_C1 is greater than that of sample Pol_C2, which is itself greater than that of sample Pol_C3, itself greater than that of sample Pol_C4, itself greater than that of sample Pol_C5. It is observed that the peak quantity Q release temperature is the same for all the samples and that the peak height depends on the polymer concentration of the sample. The more polymers in the sample, the higher the peak of quantity Q. Thus, the quantity Total_Q of sample Pol_C1 is greater than that of Pol_C2, itself greater than that of Pol_C3, itself greater than that of Pol_C4, itself greater than that of Pol_C5. Thus, quantity Total_Q depends on the polymer concentration of the porous medium.

FIG. 10b schematically illustrates, by way of non-limitative example, the evolution of the representative quantities of total organic carbon TOC, hydrocarbon compounds Total HC, carbon monoxide Total CO and carbon dioxide Total CO2 for porous medium samples consisting of a sand matrix and PET polymer, for different PET polymer concentrations in the porous medium.

The PET polymer is characterized by a quantity of total organic carbon released by a pure PET sample (without matrix) of 51.5+/−1.4%.

The top left diagram shows the evolution of the representative quantity of total organic carbon TOC (in percentage %) released by the porous medium sample as a function of the mass concentration c_pol (in percentage %) of PET in the porous medium. It is observed that this evolution is linear with a representative equation of the form:

$$TOC = 47.673 \cdot c\_pol + 0.0296$$

The top right diagram shows the evolution of the representative quantity of hydrocarbon compounds (in mg/g sample) released by the porous medium sample as a function of the mass concentration c_pol (in percentage %) of PET in the porous medium. It is observed that this evolution is linear with a representative equation of the form:

$$Total HC = 301.15 \cdot c\_pol - 0.3223$$

The bottom left diagram shows the evolution of the representative quantity of carbon monoxide (in mg/g sample) released by the porous medium sample as a function of the mass concentration c_pol (in percentage %) of PET in the porous medium. It is observed that this evolution is linear with a representative equation of the form:

$$Total CO = 135.97 \cdot c\_pol + 0.4551$$

The bottom right diagram shows the evolution of the representative quantity of carbon dioxide (in mg/g sample) released by the porous medium sample as a function of the mass concentration c_pol (in percentage %) of PET in the porous medium. It is observed that this evolution is linear with a representative equation of the form:

$$Total CO 2 = 615 \cdot c\_pol + 1.522$$

For all these evolution curves, the linear regressions have a coefficient of determination greater than 0.98, which shows a very good linearity of the evolutions of these different quantities.

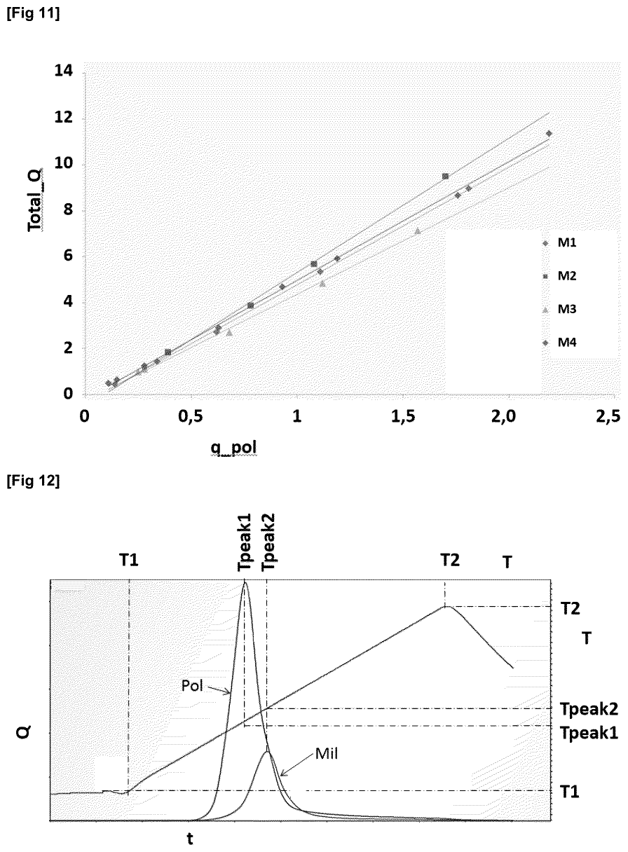

Furthermore, FIG. 11 schematically illustrates, by way of non-limitative example, relations between the total quantity measured under a peak Total_Q (that can for example correspond to Total_HC when the measurement concerns the representative quantity of hydrocarbon compounds) and the polymer quantity q_pol (the polymer is PET here) within the porous medium sample. These different relations correspond to different matrices M1, M2, M3 and M4, matrix M1 corresponding to sand, matrix M2 to clay, matrix M3 to limestone and matrix M4 to marl.

It is noted that, whatever the matrix, this relation between the total quantity measured under a peak Total_Q and polymer quantity q_pol is linear.

The relation between Total_Q and polymer quantity q_pol can be determined as follows:

$$For M 1 : Total\_Q = 5.1227 * q\_pol - 0.3389 \\ For M 2 : Total\_Q = 5.8436 * q\_pol - 0.5377 \\ For M 3 : Total\_Q = 4.6499 * q\_pol - 0.2803 \\ For M 4 : Total\_Q = 5.1705 * q\_pol - 0.191$$

Thus, once the matrix determined (by the peak temperatures), the polymers determined (by the peak temperatures) and the quantity corresponding to the area under each peak of a polymer present determined, the quantity of each of these polymers present in the porous medium can be quantified.

In step d), if at least one reference parameter (preferably several reference parameters and, more preferably, all the reference parameters) of a polymer is found in the measurements performed on the porous medium sample, the presence of this polymer in the porous medium is confirmed. Using several reference parameters (or preferably all of them) allows more accurate characterization of the presence of the polymer considered.

What is meant by “all of these reference parameters are found” or “the reference parameters are identified” is that the difference between the parameter and the corresponding parameter of the first database is less than a predetermined threshold (which may be about 10% on all the quantities and/or 10° C. (preferably 5° C.) when the reference parameters are temperatures).

Of course, the invention is not limited to the detection of a single polymer in the porous medium, and it allows several polymers to be identified. Indeed, the (at least one) reference parameters of several polymers can be identified in the porous medium, thus ensuring the presence of several polymers in the porous medium.

Thus, if at least one reference characteristic (preferably several and more preferably all of them) of a matrix is found in the measurements performed on the porous medium sample, the matrix seems to correspond to that of the porous medium. In some cases, the matrix of the porous medium may not correspond totally to the matrices of the second database. In this case, the matrix nearest thereto can be selected, which can be, for example, the matrix with the most reference characteristics found to within +/−10% in the porous medium measurements, or the matrix whose average of the differences between the measurement and the corresponding reference characteristic is the lowest.

What is meant by “all of these reference parameters are found” or “the reference parameters are identified” is that the absolute value of the difference between the parameter and the corresponding parameter of the second database is less than a predetermined threshold (which may be about 10% on all the quantities and/or 20° C. (preferably 10° C.) when the reference parameters are temperatures).

Identification of this hydrocarbon compound release peak temperature generally allows a particular polymer to be identified. Thus, if a peak of this temperature is found, up to a predetermined criterion (to within +/−10° C. for example), in the hydrocarbon compound measurement thermogram by a detector such as a FID detector from the porous medium sample, the presence of this particular polymer can be identified. The difference between the various temperatures of the peaks recorded on the hydrocarbon compound thermogram from the porous medium sample and temperature Tpeak_{HCpolymer} can for example be calculated therefore. If this difference (notably its absolute value) is less than the predefined criterion (to within 10° C. for example), the presence of the particular polymer is characterized.

Using one of the other peak temperatures (from measurement curves of the representative quantities of carbon monoxide and/or carbon dioxide during the first and/or the second heating sequence) can, as is the case with Tpeak_{HCpolymer}, help to confirm the presence of the particular polymer or to identify it.

The difference between the various temperatures of the peaks recorded on the thermogram considered from the porous medium sample and the corresponding temperature of the particular polymer can for example be calculated therefore. If this difference (its absolute value) is less than the predetermined threshold (10° C. for example), the presence of the particular polymer can be characterized or confirmed.

Using several temperatures of different thermograms makes it possible to refine the polymer determination accuracy.

Identification of the hydrocarbon compound release peak temperature generally allows a particular matrix to be identified. Thus, if a peak of this temperature is found, up to a predetermined threshold (to within +/−10° C. for example), in the hydrocarbon compound measurement thermogram by a detector such as a FID detector from the porous medium sample, the presence of this particular matrix can be identified. The difference between the various temperatures of the peaks recorded on the hydrocarbon compound thermogram from the porous medium sample and temperature Tpeak_{HCmatrix} can for example be calculated therefore. If this difference (its absolute value) is less than the predefined criterion (10° C. for example), the presence of the particular matrix is characterized.

Alternatively or additionally, the porous medium matrix can also be directly identified because it is obvious to know if the matrix concerns clay for example. In this case, the matrix corresponding to clay can be directly selected.

Using one or more of the other temperatures from the peak measurement curves (from measurement curves of the representative quantities of carbon monoxide and/or carbon dioxide during the first and/or the second heating sequence) can, as is the case with Tpeak_{HCmatrix}, help to confirm that the porous medium comprises a particular matrix or to identify it.

The difference between the various temperatures of the peaks recorded on the thermogram considered from the porous medium sample and the corresponding temperature of the particular matrix can for example be calculated therefore. If this difference (its absolute value) is less than the predetermined threshold (10° C. for example), it can be considered that the porous medium comprises the particular matrix.

Using several temperatures of different thermograms makes it possible to refine the matrix determination accuracy.

After identifying the matrix of the porous medium (either directly because clay, sand or marl has been recognized, for example, or by comparing the thermograms as explained above), the proportion of the measurements performed on the porous medium sample attributable to the polymers contained in the porous medium can then be evaluated. Indeed, the released quantities attributable to the polymers contained in the porous medium, respectively Total HC_{polymer/milieu poreux} (for the quantity of hydrocarbon compounds). Total CO_{polymer/milieu poreux} (for the quantity of carbon monoxide) and Total CO2_{polymer/milieu poreux} (for the quantity of carbon dioxide), can be deduced from the difference of these same quantities evaluated on the porous medium sample and those evaluated on the matrix alone (pure, not polluted):

$$Total {HC}_{polymer / milieu poreux} = Total {HC}_{milieu poreux} - Total {HC}_{matrix} \\ Total {CO}_{polymer / milieu poreux} = Total {CO}_{milieu poreux} - Total {CO}_{matrix} \\ Total CO {2}_{polymer / milieu poreux} = Total CO {2}_{milieu poreux} - Total CO {2}_{matrix}$$

Furthermore, the percentage of each of these products (hydrocarbon compounds, carbon monoxide and carbon dioxide) from the polymer contained in the porous medium can be deduced from these quantities.

Parameter CO_{polymer/milieu poreux} corresponds to the percentage of carbon monoxide released by the polymer contained in the porous medium during the first and second heating sequences.

Parameter CO2_{polymer/milieu poreux} corresponds to the percentage of carbon dioxide released by the polymer contained in the porous medium during the first and second heating sequences.

Parameter HC_{polymer/milieu poreux} corresponds to the percentage of hydrocarbon compounds released by the polymer contained in the porous medium during the first heating sequence.

$${CO}_{polymer / milieu poreux} = [ Total {CO}_{polymer / milieu poreux} / Total {CO}_{milieu poreux} ] * 100 ( \% ) \\ CO {2}_{polymer / milieu poreux} = [ Total {CO2}_{polymer / milieu poreux} / Total CO {2}_{milieu poreux} ] * 100 ( \% ) \\ {HC}_{polymer / milieu poreux} = [ Total {HC}_{polymer / milieu poreux} / Total {HC}_{milieu poreux} ] * 100 ( \% )$$

Thus, the presence of polymer in the porous medium can be directly deduced.

Furthermore, if a single polymer type was identified in the porous medium (by determining a single hydrocarbon compound peak temperature corresponding, to within the predefined criterion, +/−10° C. for example, to that of a polymer of the first database for example), it is possible to directly deduce the percentage of hydrocarbon compounds from this particular polymer alone. Moreover, the percentage of the polymer contained in the porous medium (or the mass of the polymer in the porous medium sample) can for example be determined from the ratio of the quantity of hydrocarbons measured during step a) in the porous medium sample to the quantity of hydrocarbon compounds released by said polymer alone (i.e. not contained in a matrix, in other words, the porous medium only contains this polymer) during the first heating sequence. Indeed, the quantity of hydrocarbon compounds from the polymer contained in the porous medium: Total HC_{polymer/milieu poreux} is known, and the quantity of hydrocarbon compounds (normalized) from a polymer alone (without matrix) Total HC_{polymer} is known. Thus, the polymer mass in the porous medium can be deduced. Since the initial mass of the porous medium sample is known, a mass ratio (or mass percentage) of polymer in the porous medium can be deduced therefrom.

When several polymers are identified, several parts of the measurement curves can be distinguished as a function of the peaks identified and attributable to the various polymers, such as areas A1 to A3 of FIG. 3 described above.

### EXAMPLES

The features and advantages of the method according to the invention will be clear from reading the application example hereafter.

FIG. 12 illustrates an example of a sand sample collected in the environment, for which the method according to the invention has been applied to characterize the presence of polymer in the porous medium.

FIG. 12 shows the representative quantity Q of hydrocarbon compounds released during an inert-atmosphere heating sequence, the heating sequence being linear between temperature T1 and temperature T2 for a predetermined length of time (about 14 minutes) for a pure polymer sample Pol (the polymer is PET here) and for a sand sample Mil.

The sand matrix has no peak of release of hydrocarbon compounds, carbon monoxide or carbon dioxide during the inert and oxidizing phases. Thus, the release peak observed on the curve of sample Mil cannot correspond to the matrix, it therefore corresponds to pollution. It is noted that the temperature of this peak Tpeak2 is very close to temperature Tpeak1, which is the temperature of the release peak observed on the curve of sample Pol of the pure PET polymer (without matrix).

The difference between temperature Tpeak 2 and temperature Tpeak1 is in a suitable tolerance interval (the tolerance interval ranging for example between 10° C. and 20° C., preferably close to 10° C.) in relation to temperature Tpeak1 of the pure polymer. Thus, the peak of the curve of porous medium sample Mil identifies the presence of the PET polymer in the porous medium. Furthermore, since no other peak is observed on the curve of porous medium sample Mil, only the PET polymer seems to pollute the sand sample. The method according to the invention allows the presence of polluting polymer in a porous medium to be clearly identified.

1. A method for characterization of the presence and/or for quantification of at least one polymer (Pol1, Pol2) in a porous medium such as a natural porous medium, characterized in that at least the following steps are carried out:
a) heating a sample of said porous medium, according to a first heating sequence in an inert atmosphere, and continuously measuring a representative quantity of hydrocarbon compounds released during said first heating sequence (HC), a representative quantity of carbon monoxide and/or a representative quantity of carbon dioxide released during said first heating sequence (CO_inert, CO2_inert),
b) heating a residue of said sample from said first heating sequence according to a second heating sequence in an oxidizing atmosphere, and continuously measuring a representative quantity of carbon monoxide and/or a representative quantity of carbon dioxide released during said second heating sequence (CO_oxyd, CO2_oxyd),
c) determining at least one parameter from at least one curve of the measured representative quantity of hydrocarbon compounds released during the first heating sequence (HC) and/or from a curve of the measured representative quantity of carbon monoxide released during the first heating sequence (CO_inert) and/or from a curve of the measured representative quantity of carbon monoxide released during the second heating sequence (CO_oxyd) and/or from a curve of the measured representative quantity of carbon dioxide released during the first heating sequence (CO2_inert) and/or from a curve of the measured representative quantity of carbon dioxide released during the second heating sequence (CO2_oxyd), and comparing said at least one parameter with at least one reference parameter of a first database relative to said at least one polymer (Pol1, Pol2),
d) from said comparison of said at least one parameter determined from at least one of said curves of one of said measured quantities with the at least one reference parameter of a first database relative to at least said polymer, characterizing the presence or the absence of said at least one polymer in the porous medium, and/or determining a quantity of said at least one polymer in said porous medium (c_pol, q_pol).

2. A method as claimed in claim 1 wherein, in step c), at least one temperature corresponding to a peak of said curve of said measured representative quantity of hydrocarbon compounds released during the first heating sequence (HC) and/or at least one temperature corresponding to a peak of said curve of said measured representative quantity of carbon monoxide released during the first heating sequence (CO_inert) and/or at least one temperature corresponding to a peak of said curve of said measured representative quantity of carbon dioxide released during the first heating sequence (CO2_inert) and/or at least one temperature corresponding to a peak of said curve of said measured representative quantity of carbon monoxide released during the second heating sequence (CO-oxyd) and/or at least one temperature corresponding to a peak of said curve of said measured representative quantity of carbon dioxide released during the second heating sequence (CO2_oxyd) is determined.

3. A method as claimed in claim 1, wherein the first database is built as follows:
I) defining several types of polymer (Pol1, Pol2), preferably at least polyethylene terephthalate, polyethylene, polyamide and/or perfluoroalkoxy,
II) for each defined polymer type, applying steps a) and b) to a sample of each defined polymer type in place of said sample of said porous medium, and determining for each defined polymer type the at least one reference parameter of the first database, the at least one reference parameter of the first database comprising at least one temperature to which the following correspond:
a peak of said curve of said measured representative quantity of hydrocarbon compounds released by said sample of said defined polymer type during said first heating sequence and/or
a peak of said curve of said measured representative quantity of carbon monoxide released by said sample of said defined polymer type during said first heating sequence and/or
a peak of said curve of said measured representative quantity of carbon dioxide released by said sample of said defined polymer type during said first heating sequence and/or
a peak of said curve of said measured representative quantity of carbon monoxide released by said sample of said defined polymer type during said second heating sequence and/or
a peak of said curve of said measured representative quantity of carbon dioxide released by said sample of said defined polymer type during said second heating sequence,
and the at least one reference parameter of the first database further preferably comprising at least:
a representative quantity of hydrocarbon compounds released by said sample of said defined polymer type during said first heating sequence and/or
a representative quantity of carbon monoxide and/or a representative quantity of carbon dioxide released by said sample of said defined polymer type during said first heating sequence and/or
a representative quantity of carbon monoxide and/or a representative quantity of carbon dioxide released by said sample of said defined polymer type during said second heating sequence,
III) adding to the first database, for each defined polymer type, the at least one reference parameter of the first database.

4. A method as claimed in claim 3, wherein step II) is repeated with several samples of each defined polymer type.

5. A method as claimed in claim 2 and in any one of claim 3 or 4 wherein, in step c), at least one of the temperatures determined in step c) (Tpeak2) for the porous medium sample is compared with at least one corresponding temperature of the at least one reference parameter of said first database (Tpeak1).

6. A method as claimed in claim 1, in step c), said at least one parameter is compared with at least one reference parameter of a second database relative to at least one matrix representative of said porous medium, the second database being built as follows:
i) defining several types of porous media matrices, preferably the matrix types comprise at least sand, marl, carbonates and clays,
ii) for each defined matrix type, carrying out steps a) and b) with a sample of each defined matrix type in place of said sample of said porous medium, and determining for each defined matrix type the at least one reference parameter of the second database, the at least one reference parameter of the second database comprising at least one temperature to which the following correspond:
a peak of said curve of said measured representative quantity of hydrocarbon compounds released by said sample of said defined matrix type during said first heating sequence and/or
a peak of said curve of said measured representative quantity of carbon monoxide released by said sample of said defined matrix type during said first heating sequence and/or
a peak of said curve of said measured representative quantity of carbon dioxide released by said sample of said defined matrix type during said first heating sequence and/or
a peak of said curve of said measured representative quantity of carbon monoxide released by said sample of said defined matrix type during said second heating sequence and/or
a peak of said curve of said measured representative quantity of carbon dioxide released by said sample of said defined matrix type during said second heating sequence,
and the at least one reference parameter of the second database preferably further comprising at least:
a representative quantity of hydrocarbon compounds released by said sample of said defined matrix type during said first heating sequence and/or
a representative quantity of carbon monoxide and/or a representative quantity of carbon dioxide released by said sample of said defined matrix type during said first heating sequence and/or
a representative quantity of carbon monoxide and/or a representative quantity of carbon dioxide released by said sample of said defined matrix type during said second heating sequence
iii) adding to the second database, for each defined matrix type, the reference parameter(s) of the second database,
and preferably, in step d), characterizing the presence or the absence of said at least one polymer in the porous medium, and/or determining a quantity of said at least one polymer in said porous medium (c_pol, q_pol) from said comparison of said at least one parameter determined from at least one of said curves of one of said measured quantities with the at least one reference parameter of the second database relative to at least one matrix representative of the porous medium.

7. A method as claimed in claim 6, wherein step ii) is repeated with several samples of each defined matrix type.

8. A method as claimed in claim 2, in step c), at least one of the temperatures determined in step c) is compared with at least one corresponding temperature of the at least one reference parameter of said second database.

9. A method as claimed in claim 5 wherein, in step c), the comparison is made by calculating at least a difference between at least one temperature corresponding to said peak of one of said curves of the quantities measured on said sample of said porous medium (Tpeak2) and the corresponding temperature of the at least one reference parameter for each defined polymer type of the first database (Tpeak1), and in step d), if, for at least one of the defined polymer types, one at least of these differences is below a predetermined threshold, one concludes to the presence of this defined polymer type in said porous medium and, in the opposite case, one concludes to the absence of this defined polymer type in said porous medium.

10. A method as claimed in claim 9 wherein, in step d), if one has concluded to the presence of said defined polymer type in said porous medium, said type of said defined polymer in said porous medium is quantified by determining a percentage of said defined polymer type in said porous medium from a ratio between the measured representative quantity of hydrocarbon compounds released during the first heating sequence in the porous medium sample and the measured representative quantity of hydrocarbon compounds released by said defined polymer type during the first heating sequence, and if one has concluded to the absence of said defined polymer type in said porous medium, a zero quantity is assigned to said defined polymer type.

11. A method as claimed in claim 1 wherein, in step c), the measured representative quantities of hydrocarbon compounds, carbon monoxide and carbon dioxide are normalized by the initial mass of the sample.

12. A method as claimed in claim 1, wherein said first heating sequence in an inert atmosphere comprises at least the following step: from a temperature ranging between 100° C. and 300° C., raising the temperature according to a temperature gradient ranging between 5 and 30° C./minute, up to a temperature ranging between 500° C. and 650° C.

13. A method as claimed in claim 1, wherein said second heating sequence in an oxidizing atmosphere comprises at least the following step: from a temperature ranging between 200° C. and 400° C., raising the temperature according to a temperature gradient ranging between 10 and 40°/minute, up to a temperature ranging between 750° C. and 950° C.
